(19) United States
(12) Patent Application Publication
(10) Pub. No.: US 2001/0030292 A1
(43) Pub. Date: Oct. 18, 2001

(54) Method of crystallising a semiconductor film

(75) Inventor: Stanley D. Brotherton, Forest Row (GB)

Correspondence Address: Jack E. Haken, Corporate Patent Counsel, U.S. Philips Corporation, 580 White Plains Road, Tarrytown, NY, 10591, US

(73) Assignee: U.S. PHILIPS CORPORATION

(21) Appl. No.: 09/828,092

(22) Filed: Apr. 6, 2001

(30) Foreign Application Priority Data
Apr. 15, 2000 (GB) 0009280.9

(51) Int. Cl. H01L 21/00; H01L 29/04; H01L 31/036; H01L 31/0376; H01L 31/20; H01L 21/84; H01L 27/01; H01L 31/0392; H01L 21/30; H01L 21/46; A61N 5/00; G21G 5/00

(52) U.S. Cl. 250/492.2; 438/166; 257/59; 257/347; 438/30; 438/459

(57) ABSTRACT

A method of crystallizing a semiconductor film (3) deposited on a supporting substrate (1,2) is disclosed together with apparatus for the same. The method comprising the steps of (a) with a laser (5), exposing each of a series of discrete regions (a to n) of the semiconductor film to one or more laser beam (4) pulses (an “exposure”); (b) monitoring the energy output of the laser (5); and (c) if the energy output of the laser (5) during an exposure of a discrete region (a to n) exceeds a predetermined threshold, re-exposing that discrete region to one or more laser beam (4) pulses.

Also disclosed is a TFT (12) manufactured by said method and active matrix device (20) comprising a row (24) and column (23) array of active elements (22), each having such a switching TFT (12).

This invention relates to a method and apparatus for crystallising a semiconductor film deposited on a supporting substrate by exposing each of a series of discrete regions of the semiconductor film to one or more laser beam pulses.

The invention further relates to a thin film transistor (TFT) having a crystalline semiconductor channel formed from such a silicon film; and to an active matrix device, especially an active matrix liquid crystal display (AMLCD), comprising a row and column array of active elements wherein each element is associated with such a TFT by connection to corresponding row and column conductors.

It is known to use pulsed laser irradiation to partially melt an amorphous (a—Si) or microcrystalline silicon film, and to allow the partially melted silicon film to cool and crystallise in order to form high quality polycrystalline silicon (poly-Si). For example, such pulsed laser irradiation is disclosed in U.S. Pat. Nos. 4,234,358, 5,591,668, 5,643,801 and 5,773,309, and PCT patent application, publication No. WO98/24118, all of which are incorporated herein by reference.

During this process, it is desirable to heat the silicon film to what is termed the “near-melt-through” condition. This is when only tiny discrete, islands of solid phase silicon remain, distributed in an otherwise entirely melted silicon film. During cooling, these tiny islands seed lateral crystalline growth enabling the formation of large grain poly-Si. The “full-melt through” condition exists when the silicon is entirely melted and no such discrete solid phase silicon islands exist. After full-melt-through as opposed to near-melt-through, the resultant crystallisation obtained is relatively poor because without the seed, there is only random nucleation resulting in fine grain silicon. The full-melt-through and near-melt-through conditions are further discussed by S D Brotherton et al., Journal of Applied Physics 82, 4086 (1997).

For optimum large grain poly-Si, the pulse energy is ideally set so as to obtain near-melt-through conditions and the level of energy required will depend amongst other things on the film thickness and also the presence of any surface films which may influence surface reflectivity, and substrate temperature. Unfortunately however, the process window to obtain near-melt-through as opposed to full-melt-through is relatively narrow, and inadvertent full-melt-through may occur resulting in poor crystallisation, especially where relatively thin silicon films are irradiated.

In manufacturing equipment in which a thin laser beam is scanned across a substrate, either by movement of the beam over a stationary substrate or, as is more conventional, movement of the underlying substrate, this can result in a line of TFTs across a wafer having inferior electrical characteristics attributable to the poor crystallinity of their respective channels.

Thus, it is an object of the present invention to provide such a method of manufacturing a semiconductor film in which the effects of inadvertent full-melt-through are mitigated.

In accordance with a first aspect of the present invention, there is provided a method of crystallising a semiconductor film deposited on a supporting substrate comprising the steps of:

(a) with a laser, exposing each of a series of discrete, possibly overlapping, regions of the semiconductor film to one or more laser beam pulses (an “exposure”), preferably intended to heat the discrete regions to a near-melt-through condition;

(b) monitoring the energy output of the laser; and

(c) if the energy output of the laser during an exposure of a discrete region exceeds a predetermined threshold (an “over-exposure”), re-exposing that discrete region to one or more laser beam pulses (a “re-exposure”).

The predetermined threshold may be set at or above the energy output required to heat a discrete region to a full-melt-through condition, for example, at between 105% and 115% or 107% and 110% of the energy output associated with the intended exposure. Also, in the event of an over-exposure of a discrete region to a full-melt-through condition, that discrete region is preferably allowed to completely solidify prior to re-exposure.

The inventor has realised that random fluctuations in pulse energy set against the narrow process window for obtaining near-melt-through will result in occasional inadvertent full-melt-through despite best efforts to avoid this happening. The method of the present invention in effect repairs regions of poor crystallinity attributable to full-melt-through by repeating the laser pulse crystallisation process if the power fluctuations exceed a predetermined level.

The discrete regions of the semiconductor film may be defined by the shape of a long thin laser beam produced by a laser capable of being scanned over the semiconductor film in a stepped fashion. In such an arrangement, the laser may sequentially expose discrete regions of the semiconductor film and in the event of an over-exposure of a discrete region, that discrete region may be re-exposed prior to stepping to or exposing an adjacent discrete region, i.e. providing a uni-directional scan over the semiconductor film.

Also provided is apparatus for crystallising a semiconductor film comprising a supporting substrate for receiving a semiconductor film; a laser for exposing each of a series of discrete regions of the semiconductor film; and a control unit for monitoring the energy output of the laser apparatus.

In accordance with a second aspect of the present invention, there is provided a method of manufacturing a thin film transistor (TFT) comprising source and drain electrodes joined by a semiconductor channel, a gate insulating layer and a gate electrode, wherein the semiconductor channel was formed from a semiconductor film crystallised by a method according to the first aspect of the present invention; and furthermore, a TFT manufactured by the same.

Lastly, in accordance with a third aspect of the present invention, there is an active matrix device comprising a row and column array of active elements wherein each element is associated with a switching TFT according to the second aspect of the present invention and connected to corresponding row and column conductors.

Methods of manufacturing a TFT according to the present invention and an AMLCD incorporating TFTs manufactured by the same will now be described, by way of example only, with reference to the accompanying figures in which:

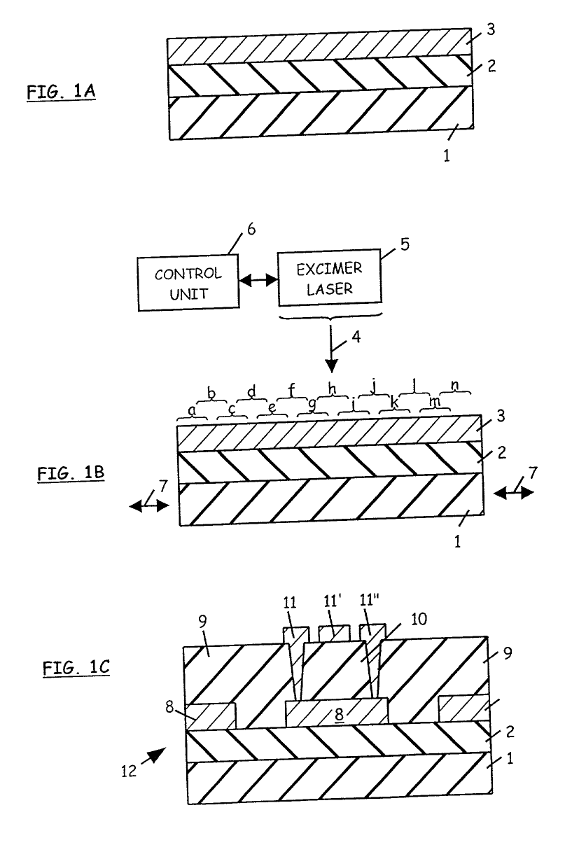

FIGS. 1A to 1C illustrate a method of manufacturing a TFT structure according to the present invention; and

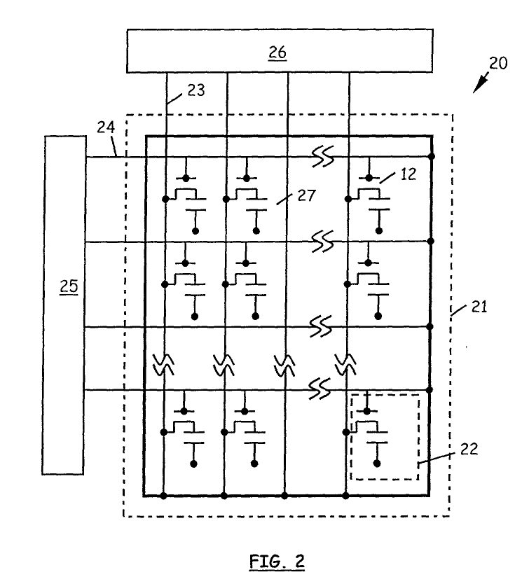

FIG. 2 shows, schematically, a AMLCD incorporating TFTs manufactured by the method illustrated in FIGS. 1A to 1C.

Referring to FIG. 1A to 1C, a method of manufacturing a TFT according to the present invention is described below.

On a borosilicate glass substrate 1 such as Corning Co.'s No 1737, an insulating silicon oxide film 2 is deposited on top of the glass substrate to a thickness of between 50 nm to a few hundred nm, say 400 nm (4000Å). An a—Si film 3 is then deposited on top of the silicon oxide film also by plasma CVD to a thickness of approximately 40 nm (400Å). In order to crystallise the silicon film 3, a thin pulsed laser beam 4 is sequentially stepped across the silicon film, each step relating to a different region of the silicon film denoted in FIG. 1C as overlapping regions a to n. For example, the pulsed laser beam may be stepped sequentially from a to n, pulsing 20 shots per region at 300 mJ/cm2 with the intention of heating each region to a near-melt-through condition.

The pulsed laser beam is provided by a excimer laser 5 controlled by a control unit 6 and, in accordance with the present invention, the energy output of the laser apparatus is monitored by the control unit when each region of the semiconductor film is exposed to a laser beam pulse or pulses.

If the energy output of the laser during exposure exceeds 107% of the expected level of energy output for the intended exposure and at which full-melt-through can be presumed to have occurred, that region is allowed to cool and re-solidify, and is then re-exposed whereby it is re-heated to a near-melt-through condition. Of course, this cycle can be repeated such that if a power fluctuation during the re-exposure is likely to leave the region in a full-melt-through condition, it can be allowed to cool and further re-exposed.

The pulse frequency of conventional laser pulse annealing is typically less than 300 Hz, corresponding to an available cooling time between over-exposure and re-exposure of at least approximately 3 ms. As this far exceeds the time required for re-solidification of a film, typically around 100 ns, allowing the film to cool after overexposure will not normally delay re-exposure.

Conceivably however, where the laser pulse frequency is sufficiently high as to leave a previously over-exposed region with residual heat energy at re-exposure, the subsequent re-exposure may be of a reduced energy compared to the energy intended for the initial exposure. That is, utilising the residual heat energy in the over-exposed region in order to achieve near-melt-through at a second or later attempt. As an alternative, an over-exposed region may be re-exposed only after at least one other region has been exposed. This would provide an over-exposed region with more time to cool prior to re-exposure without significantly lengthening the overall process time. For example, with reference to FIG. 1B, the exposure sequence a, b, c(over-exposure), d, e, f, c(re-exposure), g, h and so on.

The silicon film 3 is doped, typically either before laser annealing or after gate definition, and etched to form device islands 8 using conventional doping and mask etching techniques. A silicon oxide gate insulating layer 9 is deposited using plasma CVD and patterned to form a gate insultor 10; and metal source 11, gate 11′ and drain 11″ electrodes are provided, resulting in the TFT structure 12 shown schematically in FIG. 1C.

As mentioned previously, the level of energy required to heat each region to a near-melt-through condition will vary depending on factors such as film thickness and also the presence of any surface films. It may therefore be necessary to determine the level of energy required for any given semiconductor process on a case by case basis, as would be readily done by a person skilled in the art of semiconductor manufacture.

Referring to FIG. 2, an AMLCD is shown, schematically, incorporating TFTs manufactured by the method illustrated in FIGS. 1A to 1C. The AMLCD 20 comprises an display area 21 consisting of m rows (1 to m) and n columns (1 to n) of identical picture elements 22. Only a few of the picture elements are shown for simplicity whereas in practice, the total number of picture elements (m×n) in the display area may be 200,000 or more. Each picture element 22 has a picture electrode 27 and associated therewith a switching TFT 10 of the type manufactured by the method illustrated in FIGS. 1A to 1C, and which serves to control the application of data signal voltages to the picture electrode. The switching TFTs have common operational characteristics and are each arranged adjacent to their associated picture element with their respective drain being connected to the picture electrode. The sources of all switching TFTs associated with one column of picture elements are connected to a respective one of a set of parallel column conductors 23 and the gates of all switching TFTs associated with one row of picture elements are connected to a respective one of a set of parallel row conductors 24. The TFTs are controlled by gating signals provided via the row conductors by row driver circuitry 25 external to the display area 21. Similarly, the TFTs associated with picture elements in the same column are provided with data signal voltages for the picture electrodes by column driver circuitry 26 also external to the display panel. Of course, the configuration and operation of such AMLCD picture elements is well known and accordingly will not be elaborated upon here further.

The specific considerations for the practical manufacture of thin film transistors and of active matrix devices incorporating the same will be apparent to those skilled in the art, and the considerations which should be applied for existing transistor designs should also be applied for design of a transistor in accordance with the invention. The precise process conditions which may be appropriate have not been described in this text, as this is a matter of normal design procedure for those skilled in the art.

1. A method of crystallising a semiconductor film deposited on a supporting substrate comprising the steps of:
(a) with a laser, exposing each of a series of discrete regions of the semiconductor film to one or more laser beam pulses (an “exposure”);
(b) monitoring the energy output of the laser; and
(c) if the energy output of the laser during an exposure of a discrete region exceeds a predetermined threshold (an “over-exposure”), reexposing that discrete region to one or more laser beam pulses (a “re-exposure”).

2. A method according to claim 1 wherein each exposure is intended to heat a discrete region to a near-melt-through condition.

3. A method according to claim 1 or claim 2 wherein the predetermined threshold is set at between 105% and 115% of the energy intended for an exposure.

4. A method according to claim 3 wherein the predetermined threshold is set at between 107% and 110% of the energy intended for an exposure.

5. A method according to any claim 1 or claim 2 wherein the predetermined threshold is set at or above the energy output required to heat a discrete region to a near full-melt-through condition.

6. A method according to claim 5 wherein in the event of an over-exposure of a discrete region, that discrete region is allowed to completely solidify prior to re-exposure.

7. A method according to any preceding claim wherein at least some of the discrete regions of the semiconductor film overlap.

8. A method according to any preceding claim wherein the laser produces a long thin laser beam capable of being scanned over the semiconductor film in a stepped fashion, thereby defining the discrete regions of the semiconductor film.

9. A method according to claim 8 wherein the laser beam is stepped over the semiconductor film, sequential exposing discrete regions of the semiconductor film wherein in the event of an over-exposure of a discrete region, that discrete region is re-exposed prior to stepping to an adjacent discrete region.

10. A method according to claim 8 wherein the laser beam is stepped over the semiconductor film, sequentially exposing discrete regions of the semiconductor film wherein in the event of an over-exposure of a discrete region, that discrete region is re-exposed prior to exposing an adjacent discrete region.

11. A method of crystallising a semiconductor film as hereinbefore described with reference to accompanying FIGS. 1A to 1C.

12. A semiconductor film crystallised by a method according to any preceding claim.

13. A method of manufacturing a thin film transistor (TFT) comprising source and drain electrodes joined by a semiconductor channel, a gate insulating layer and a gate electrode, wherein the semiconductor channel was formed from a semiconductor film crystallised by a method according to claims 1 to 11.

14. A method of manufacturing a thin film transistor (TFT) substantially as hereinbefore described with reference to the accompanying figures.

15. A TFT manufactured by a method according to claim 13 or claim 14.

16. An active matrix device comprising a row and column array of active elements wherein each element is associated with a switching TFT according to claim 15 connected to corresponding row and column conductors.

17. Apparatus for crystallising a semiconductor film comprising a supporting substrate for receiving a semiconductor film; a laser for exposing each of a series of discrete regions of the semiconductor film to one or more laser beam pulses; and a control unit for monitoring the energy output of the laser apparatus.

18. Apparatus for crystallising a semiconductor film by a method according to claims 1 to 11 comprising a supporting substrate for receiving a semiconductor film; a laser for exposing each of a series of discrete regions of the semiconductor film to one or more laser beam pulses; and a control unit for monitoring the energy output of the laser apparatus.
